(19) United States
(12) Patent Application Publication
(10) Pub. No.: US 2019/0380715 A1
(43) Pub. Date: Dec. 19, 2019

(54) ENDOSCOPIC CUFFS

(71) Applicant: SHAARE ZEDEK SCIENTIFIC LTD., Jerusalem (IL)

(72) Inventors: Eran GOLDIN, Tel Aviv (IL); Tomer ADAR, Ramat Hasharon (IL)

(73) Assignee: SHAARE ZEDEK SCIENTIFIC LTD., Jerusalem (NL)

(21) Appl. No.: 16/465,514

(22) PCT Filed: Dec. 4, 2017
(86) PCT No.: PCT/IL2017/051311

§ 371 Date: May 30, 2019

Related U.S. Application Data
(60) Provisional application No. 62/430,041, filed on Dec. 5, 2016.

(51) Int. Cl. A61B 17/128; A61B 17/122

(52) U.S. Cl. CPC A61B 17/1285; A61B 1/00135; A61B 17/1227

(57) ABSTRACT

An endoscopic cuff having an outer surface configured to allow access of the cuff to tight tissue locations, wherein the cuff has a first collapsed configuration and a second expanded configuration; wherein the expanded configuration is configured to allow passage of one or more endoscopic accessory devices through the at least one longitudinal bore.

### TECHNICAL FIELD

The present disclosure generally relates to the field of endoscopy, more particularly to endoscopic cuffs configured to provide working channels for irrigation, introduction of medical devices, and/or tissue extraction.

### BACKGROUND

Endoscopes are presently used for screening, diagnostic, and therapeutic purposes.

There are many different uses for endoscopes, and while the general design of different models is similar, some variations exist to optimize the performance of the endoscope for its intended purpose.

The endoscope may include and/or allow passage of one or more diagnostic or treatment devices.

Conventional endoscopes, such as the endoscope illustratively depicted in FIG. 1, have an endoscope probe 100 connected at its proximal end to a handle (not shown). Probe 100 is adapted to be inserted into a patient's body cavity to perform selected therapeutic, diagnostic, and/or screening procedures. Probe 100 typically contains an imaging system 110 having optical fibers, or the like, extending along the length of the probe, a light guide 120, an air/water spray nozzle 130 and a working channel 140.

A limiting constraint in designing endoscopes is that the diameter of the endoscope must be less than the diameter of the body cavity through which the endoscope must travel. While the inside cavity may be much larger than the scope, the latter must pass through specific openings, whose diameters may be a limiting factor to the scopes' diameter. Similarly, the ability of a patient to tolerate an endoscope is related to its diameter.

Resultantly, the medical procedures that can be performed using an endoscope are generally limited by the working channels' number and diameter, or to devices which are mounted at the tip of the scope. For example, when using a single channel endoscope, the operator can use only one through-the-scope instrument, and when such an instrument is placed within the working channel, the ability to irrigate through the working channel may be limited. Furthermore, when there is need to extract content (such as, but not limited to, tissue and foreign bodies) the diameter of the working channel may not be sufficient to permit extraction through the scope, and extraction of the entire scope while grasping the desired content (e.g. by net) is required.

In addition to limiting the size of content that can be extracted through the scope, the working channel's diameter also limits the diameter of instruments that can be inserted through it.

Dual channel endoscopes have been developed, which have the ability to introduce additional accessory devices. However, their external diameter is larger than standard scope models, and may be associated with difficulty and discomfort when introduced into the body cavity.

There, therefore, remains a need for low-cost and disposable means for adding working channels to conventional endoscopes, with larger diameter while leaving their external diameter minimally affected during scope insertion.

### SUMMARY

The present disclosure is directed to endoscopic cuffs configured to be draped over endoscopic probes, the cuff including working channels allowing introduction of additional and larger endoscopic accessory devices, medical instruments, imaging probes, therapeutics and extractions of larger content, for procedures within an organ's lumen, organ wall, and/or outside the organ to which the endoscope was introduced and/or advanced. According to some embodiments, the endoscope is adapted for gastrointestinal (GI) use, such as, but not limited to, esophagogastroduodenoscopy, enteroscopy, colonoscopy, sigmoidoscopy, anoscopy, choledochectomy, and the like. Each possibility is a separate embodiment. According to some embodiments, the endoscope is adapted for non-GI use such as, but not limited to, nasal endoscopy, rhinoscopy, laryngoscopy, bronchoscopy, otoscopy, cystoscopy, colposcopy and hysteroscopy, laparoscopy, arthroscopy, thoracoscopy, mediastinoscopy, and the like. Each possibility is a separate embodiment. According to some embodiments, the endoscope is a forward viewing endoscope, but may also be adapted for non-forward viewing endoscopes, such as side-viewing scopes (e.g. those used for endoscopic retrograde cholangiopancreatography (ERCP)), wide angle scopes, anterior oblique and other forms of endoscopes including endoscopic ultrasound (E-US) scopes.

Advantageously, during introduction into a body cavity, the working channels of the cuff, draped over the probe, are collapsed during insertion, such that only a minimal increase in the overall outer diameter is incurred. Once reaching its target area, the working channels can be utilized, for example by being expanded by instruments, air/gas/liquid insufflation or irrigation and provide additional working channel(s) allowing access of endoscopic accessory devices, medical instruments, imaging probes, irrigation, content extraction and more.

According to some embodiments, the cuff may be a single use disposable and may be wrapped around the probe prior to each use without preceding knowledge as to whether additional working channels are required/desired, thereby allowing decision making during procedures. This may be particularly advantageous, when the endoscope is used for diagnostic purposes in that treatment, if realized to be required, may be provided immediately without necessitating scope extraction and reinsertion of the endoscope probe, which may prolong procedure and anesthesia time, and may be associated with discomfort and complications.

As a further advantage, the cuff may be formed of a single, one-piece material. This allows for low production costs, e.g. by molding using a single cast and even 3-D printing. According to some embodiments, the part of the cuff forming the one or more longitudinal bores is made of a single, one-piece material.

In addition, due to being formed of a single one-piece material, the need for joining materials or sheaths is obviated. This is of utmost importance in that the Achilles' heel of endoscopic cuffs and sheaths made of two or more joined together materials is the joint, which, on the one hand, may unravel and thus require replacement, and, on the other hand, may introduce unevenness, which may cause injury to delicate internal tissues.

As a further advantage, a specialized net for content retrieval has been developed, using a combination of an expandable net and an inflatable balloon designed to facilitate passage through the external channel of the cuff for content extraction (including, but not limited to, polyps, tissue biopsies and foreign bodies). The cuff may further include a large opening at its proximal end, shaped and sized to allow the extracted matter to exit the cuff.

According to some embodiments, there is provided an endoscopic access system comprising a cuff having an outer surface configured to allow access of the cuff to tight tissue locations, an inner surface configured to completely or partially circumferentially encase, engage, enclose or sheath an endoscopic probe, at least one longitudinal bore formed between the inner and outer surfaces, and an attachment assembly configured to engage a distal tip of the endoscopic probe, thereby preventing rearward movement of the cuff relative to the endoscopic probe.

According to some embodiments, the cuff has a first collapsed configuration and a second expanded configuration; wherein in its collapsed configuration the outer diameter of the cuff engaging the endoscopic probe is below 13 mm, below 12 mm or below 11 mm and wherein the expanded configuration is configured to allow passage of one or more endoscopic accessory devices through the at least one longitudinal bore.

According to some embodiments, the cuff may be formed from a one-piece material thereby obviating a need for joining materials.

According to some embodiments, the endoscopic cuff may be configured to partially or completely encircle the endoscopic probe. According to some embodiments, the endoscopic cuff may be configured to completely encircle parts of the endoscopic probe while other parts are partially encircled.

According to some embodiments, the cuff may assume its expanded configuration when one or more endoscopic accessory devices are passed through the at least one longitudinal bore.

According to some embodiments, the cuff may assume its expanded configuration when a guide is used to advance instruments through the at least one longitudinal bore. This may also serve to guide the instruments directly through the channel, preventing the instruments from looping and from being pushed against the channels side walls.

According to some embodiments, the cuff may assume its expanded configuration when one or more endoscopic accessory devices are passed through the at least one longitudinal bore with a cap placed on its end. This may serve to guide the instruments directly through the channel, preventing the instruments from looping and from being pushed against the channels' side walls.

According to some embodiments, the cuff may resume its collapsed configuration when the one or more endoscopic accessory devices are pulled out of the at least one longitudinal bore. According to some embodiments, each longitudinal bore may resume a collapsed/expanded configuration separately, e.g. as a result of passing an instrument through the longitudinal bore. According to some embodiments, all longitudinal bores may resume a collapsed/expanded configuration in a coordinated manner (e.g. simultaneously or sequentially).

According to some embodiments, the diameter of the cuff may be enlarged segmentally during passage of the one or more endoscopic accessory devices.

According to some embodiments, the cuff may be made from a shape memory alloy or a shape memory polymer.

According to some embodiments, the attachment assembly may include a tapered portion at a distal tip of the cuff; such that the circumference of the distal tip is smaller than the circumference of the distal end of the endoscopic probe.

According to some embodiments, the attachment assembly may include an attachment element configured to grasp or attach to a distal end of the endoscopic probe.

According to some embodiments, when in its collapsed configuration, the outer diameter of the cuff encaging/engaging the endoscopic probe may be below 15 mm, below 14 mm, below 13 mm, below 12 mm, below 11 mm or below 10 mm.

According to some embodiments, the at least one longitudinal bore may include a smoothing layer configured to allow essential smooth passage of endoscopic accessory devices therethrough.

According to some embodiments, the cuff may include a plurality of pores opening into the at least one longitudinal bore, the plurality of pores containing a smoothening fluid.

According to some embodiments, the diameter of the pores may increase when the cuff assumes its expanded position, thereby causing the smoothening fluid to spread on the wall of the at least one longitudinal bore.

According to some embodiments, the at least one longitudinal bore may be sized and shaped to allow withdrawal of large tissue specimens, bowel content and/or foreign bodies therethrough, without requiring withdrawal of the endoscopic probe.

According to some embodiments, the at least one longitudinal bore may include a polyp retriever device.

According to some embodiments, the polyp retriever device may include an inflatable balloon configured to enlarge at least one longitudinal bore prior to retrieving a polyp, tissue, bowel content or a foreign body therethrough.

According to some embodiments, the system may further include a large chamber and/or opening at its proximal end, the large opening configured to allow withdrawal of a polyp, tissue, bowel content and/or a foreign body collected through the at least one longitudinal bore.

According to some embodiments, the cuff may further include a large opening at its proximal end, the large opening configured to allow insertion and use of instruments and devices whose diameter is larger than a working channel of the endoscopic probe.

According to some embodiments, the large opening may include a hood configured to cover the large chamber and/or opening.

According to some embodiments, the large opening may include a scaffold configured for mounting and inserting endoscopic equipment.

According to some embodiments, the cuff may further include a handle anchor having at least one access port, the at least one access port configured to provide access of the one or more endoscopic accessory devices to the at least one longitudinal bore.

According to some embodiments, expansion of the cuff increases an outer diameter of the cuff while leaving an inner diameter of the cuff essentially unaffected, thereby increasing the diameter of the at least one longitudinal bore.

According to some embodiments, the cuff may further include a positioning element at its distal end, the positioning element configured to ensure that the cuff is correctly positioned on the endoscopic probe.

According to some embodiments, the cuff may include at least two longitudinal bores. According to some embodiments, at least one of the least two longitudinal bores may be larger than the remainder of the at least two longitudinal bores.

According to some embodiments, there is provided an endoscopic cuff including an outer surface configured to allow access of the cuff to tight tissue locations; an inner surface configured to circumferentially encase, engage, enclose or sheath an endoscopic probe; at least one longitudinal bore formed between the inner and outer surfaces; an attachment assembly configured to engage the distal tip of the endoscopic probe, thereby preventing rearward movement of the cuff relative to the endoscopic probe; and a polyp retriever device configured for insertion through the at least one longitudinal bore, the polyp retriever device comprising an inflatable balloon configured to enlarge the at least one longitudinal bore and/or narrow orifices prior to retrieving a polyp, tissue, and/or a foreign body therethrough.

According to some embodiments, the cuff has a first collapsed configuration and a second expanded configuration.

According to some embodiments, in its collapsed configuration, the outer diameter of the cuff encaging/engaging the endoscopic probe is below 15 mm, below 14 mm, below 13 mm, below 12 mm, below 11 mm or below 10 mm.

According to some embodiments, the cuff may further include a positioning element at its distal end, the positioning element configured to ensure that the cuff is correctly positioned on the endoscopic probe.

According to some embodiments, the cuff may further include a large opening at its proximal end, the large opening configured to allow withdrawal of the polyp, the tissue, and/or the foreign body collected through the at least one longitudinal bore. According to some embodiments, the large opening comprises a hood configured to cover the large opening.

According to some embodiments, the cuff may include at least two longitudinal bores. According to some embodiments, at least one of the least two longitudinal bores is larger than the remainder of the at least two longitudinal bores.

According to some embodiments, there is provided an endoscope assembly including an endoscope probe and a cuff, the cuff including an outer surface configured to allow access of the cuff to tight tissue locations, an inner surface configured to circumferentially encase, engage, enclose or sheath an endoscopic probe, at least one longitudinal bore formed between the inner and outer surfaces, an attachment assembly configured to engage the distal tip of the endoscopic probe, thereby preventing rearward movement of the cuff relative to the endoscopic probe; and a polyp retriever device configured for insertion through the at least one longitudinal bore, the polyp retriever device comprising an inflatable balloon configured to enlarge the at least one longitudinal bore prior to retrieving the polyp therethrough.

According to some embodiments, the cuff has a first collapsed configuration and a second expanded configuration; wherein in its collapsed configuration the outer diameter of the cuff encaging/engaging the endoscopic probe is below 15 mm, below 14 mm, below 13 mm, below 12 mm, below 11 mm or below 10 mm.

According to some embodiments, the assembly may further include an endoscopic clipping device, the endoscopic clipping device comprising at least two graspers configured to grasp tissue, a clip storage/cartridge/magazine comprising at least one clip, and a clip release mechanism configured to release a clip from the clip storage/cartridge/magazine when activated.

According to some embodiments, there is provided a guide probe configured to enlarge a longitudinal bore of an endoscopic cuff, wherein a distal end of the guide probe is blunt and comprises an inflatable/deflectable balloon. According to some embodiments, the guide probe may further include a plurality of pores configured to facilitate lubrication upon insertion. According to some embodiments, the guide probe may serve to guide the instruments directly through the channel, preventing the instruments from looping and from being pushed against the channels' side walls.

According to some embodiments, there is provided an endoscope assembly comprising an endoscope probe and a cuff, the cuff including an outer surface configured to allow access of the cuff to tight tissue locations, an inner surface configured to circumferentially encase/engage/enclose/sheath an endoscopic probe, at least one longitudinal bore formed between the inner and outer surfaces, an attachment assembly configured to engage the distal tip of the endoscopic probe, thereby preventing rearward movement of the cuff relative to the endoscopic probe, and an endoscopic clipping device including at least two graspers configured to grasp tissue, a clip storage/cartridge/magazine comprising at least one clip and a clip release mechanism configured to release a clip from the clip storage when activated. According to some embodiments, the clip storage/cartridge/magazine comprises at least two clips.

According to some embodiments, the endoscope assembly may further comprise a guide wire configured to guide the endoscope probe, the polyp retriever, the grasper or other endoscopic accessory device within the at least one longitudinal bore.

According to some embodiments, there is provided a clipping device comprising at least two graspers, at least two clips and a clip release mechanism configured to release a clip from the clip storage/cartridge/magazine when activated. According to some embodiments, the clip release mechanism includes a guide configured to push a distal end of the at least two clips over an underlying cover, thereby releasing the distal clip from the clip storage/cartridge/magazine.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

### BRIEF DESCRIPTION OF THE DRAWINGS

Examples illustrative of embodiments are described below with reference to figures attached hereto. In the figures, identical structures, elements or parts that appear in more than one figure are generally labeled with a same numeral in all the figures in which they appear. Alternatively, elements or parts that appear in more than one figure may be labeled with different numerals in the different figures in which they appear. Dimensions of components and features shown in the figures are generally chosen for convenience and clarity of presentation and are not necessarily shown in scale. The figures are listed below.

FIG. 1 shows a distal end of a conventional endoscope probe;

FIG. 2A shows a front view of an endoscopic cuff draped around an endoscopic probe in its collapsed configuration; according to some embodiments;

FIG. 2B shows an endoscopic cuff draped around an endoscopic probe in its fully expanded configuration; according to some embodiments;

FIG. 2C shows an endoscopic cuff draped around an endoscopic probe having a single longitudinal bore in an expanded configuration, according to some embodiments;

FIG. 3A shows a front view of an endoscopic cuff draped around an endoscopic probe, having different sized bores, in its collapsed configuration; according to some embodiments;

FIG. 3B shows an endoscopic cuff draped, having different sized bores, around an endoscopic probe in its expanded configuration; according to some embodiments;

FIG. 4A shows a front view of an endoscopic cuff draped around an endoscopic probe having a single bore in its collapsed configuration; according to some embodiments;

FIG. 4B shows an endoscopic cuff draped around an endoscopic probe having a single bore in its expanded configuration; according to some embodiments;

FIG. 5 shows a side view of an endoscopic cuff draped around an endoscopic probe, according to some embodiments;

FIG. 6 shows a side view of a proximal end of an anchoring element of an endoscopic cuff draped around the handle of an endoscopic probe, according to some embodiments;

FIG. 7 shows a side view of a proximal end of an endoscopic cuff including entry channels and a large opening, draped around the handle of an endoscopic probe, according to some embodiments;

FIG. 8 shows the distal end of an endoscopic cuff including a polyp retriever; according to some embodiments;

FIG. 9 shows the distal end of an endoscopic cuff including a polyp retriever with an inflatable balloon; according to some embodiments;

FIG. 10A-10G show an endoscopic clipping device configured for use with an endoscope cuff; according to some embodiments.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure. Additionally, it is to be explicitly understood that any combination of any one or more of the disclosed embodiments may be applicable and is within the scope of the disclosure.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” or “comprising”, when used in this specification, specify the presence of stated features, integers, steps, operations, elements, or components, but do not preclude or rule out the presence or addition of one or more other features, integers, steps, operations, elements, components, or groups thereof.

The present disclosure generally relates to the field of endoscopy, more particularly to endoscopic cuffs configured to provide working channels for introduction of medical devices, imaging probes, therapeutics, and content retrieval.

According to some embodiments, the cuff may be used with endoscopic probes of various sizes, such as, but not limited to, colonoscopes, gastroscopes, side-viewing scopes, endoscopic ultrasound scopes, adult and pediatric, rigid and flexible scopes single and multi-channel scopes. According to some embodiments, the cuff may fit and/or be suitable for use with non-GI scoping instrumentation.

According to some embodiments, there is provided an endoscopic cuff including an outer surface, forming at least one longitudinal bore. According to some embodiments, the cuff may further include an inner surface in which case the longitudinal bore may be formed between the inner and outer surfaces.

As used herein, the terms “cuff”, “sheath” and “overtube” may be used interchangeably and refer to a sheath-like layer of material configured to cover or be draped over an endoscopic probe. The endoscopic probe may be completely or partially enclosed by the external cuff.

As used herein the terms “bore” and “channel” may be used interchangeably and refer to a hollow compartment formed along the length of the cuff (along its longitudinal axis) and suitable for serving as a working channel, irrigation, and/or for passage of endoscopic accessory devices, medical instruments, imaging probes, and/or therapeutics, when expanded. According to some embodiments, the bore may be formed as channels in the (single) material from which the bore is formed. According to some embodiments, the material forming the cuff may be flexible.

According to some embodiments, the working channel may extend along the entire length of the endoscope probe. According to some embodiments, the working channel(s) may extend along part of the length of the endoscope probe. According to some embodiments, the working channel(s) may be essentially parallel to the working channel of the endoscope probe. According to some embodiments, the working channel(s) may be angled relative to the endoscope probe, at least along part of its length. As a non-limiting example, the working channel(s) may exit in an upward angle, thereby allowing better retraction.

As used herein, the terms “patient” and “subject” may be interchangeably used and may refer to any subject undergoing an endoscopic procedure.

According to some embodiments, the cuff may include more than one longitudinal bore, such as 2, 3, 4, 5 or more longitudinal bores. Each possibility is a separate embodiment. According to some embodiments, the cuff may include a plurality of longitudinal bores. According to some embodiments, the cuff may include a single longitudinal bore. According to some embodiments, the cuff may include at least two longitudinal bores. According to some embodiments, the at least two bores may have a same size and/or shape. According to some embodiments, the at least two bores may have a different size and/or shape.

According to some embodiments, each of the longitudinal bores may be circumferentially spaced around the endoscopic probe, thereby permitting a combination of endoscopic accessory devices to be used in co-operation with each other to perform a medical procedure. This may be of particular importance when performing complex procedures, such as, but not limited to, endoscopic submucosal dissection (ESD).

According to some embodiments, all or some of the longitudinal bores may terminate in a distal end opening. According to some embodiments, at least one of the longitudinal bores may terminate in a distal end opening. According to some embodiments, all or some of the longitudinal bores may include one or more apertures/openings positioned proximally to the distal end, such as, but not limited to, 1-10 cm from the distal end of the cuff, which one or more apertures/openings are configured to allow accessory devices, medical instruments, imaging probes therapeutics, to exit/be retracted therethrough. According to some embodiments, at least one of the longitudinal bores may include one or more apertures/openings positioned proximally to the distal end thereof. According to some embodiments, all or some of the longitudinal bores may have a sealed distal end. According to some embodiments, at least one of the longitudinal bores may have a sealed distal end.

According to some embodiments, the outer surface is configured to allow essentially smooth access of the cuff to tight tissue locations. That is, the outer surface of the cuff may, according to some embodiments, be made of a lubricious material configured to allow smooth passage, e.g. a material allowing the endoscopic probe, covered by the cuff, to essentially glide in to a desired tissue location, i.e. the rectum of a subject. Additionally or alternatively, the outer surface may be covered by an additional lubricious layer configured to allow smooth access of the endoscopic probe, covered by the cuff. According to some embodiments, the additional layer may be an integral part of the cuff, be provided with the cuff or applied on the cuff prior to use (e.g. after having been applied on the probe, but prior to insertion).

According to some embodiments, the inner surface of the cuff is configured to circumferentially encase/engage/enclose/sheath an endoscopic probe. According to some embodiments, the inner surface of the cuff may be made of a material configured to allow the cuff to be easily applied on the probe. Additionally or alternatively, the inner surface may be covered by an additional layer configured to allow the cuff to be easily applied on the probe. According to some embodiments, the additional layer may be an integral part of the cuff, be provided with the cuff or applied on the cuff prior to use. Each possibility is a separate embodiment.

According to some embodiments, the inner surface of the cuff may be made of a material configured to ensure friction compression fit of the cuff to the probe, i.e. to prevent the cuff from moving relative to the probe once applied thereon. Additionally or alternatively, the inner surface may be covered by an additional layer configured to ensure friction compression fit of the cuff to the probe. According to some embodiments, the additional layer may be an integral part of the cuff, be provided with the cuff or applied on the cuff prior to use. Each possibility is a separate embodiment.

According to some embodiments, the cuff may be sized and shaped to prevent the cuff from moving relative to the endoscope probe once applied thereon.

According to some embodiments, the cuff may include an attachment assembly configured to engage the distal tip of the endoscopic probe, thereby preventing the probe from passing through the distal end of the cuff and/or preventing the cuff from retracting/rolling back during forward movement/insertion of the probe into the body cavity. According to some embodiments, the attachment assembly may be in a shape of the cuff. For example, the cuff may be tapered in circumference such that the circumference at the distal end of the cuff is somewhat smaller than the circumference of the distal end of the probe. According to some embodiments, the attachment assembly may be an element such as a clip, a hook, a button or any other suitable element affixed to or formed with the distal end of the endoscopic probe and configured to prevent the probe from passing through the distal end of the cuff and/or preventing the cuff from retracting/rolling back during forward movement/insertion of the probe into the body cavity.

According to some embodiments, the cuff may include a positioning element configured to ensure that the cuff is correctly positioned on the endoscope probe. This is crucial in order to ensure that the accessory device passed through the longitudinal bore of the cuff will exit the cuff at the correct position, i.e. the position visualized by the imaging system of the endoscope probe. Even small deviations may cause perforations of the surrounding tissue and thus cause great harm. According to some embodiments, the positioning element may be a small balloon, tab, or other similar element visualized by the imaging system, yet without interfering with the imaging needed to perform the procedure of interest. Additionally or alternatively, the cuff and/or its distal assembly may include a positioning sensor configured to provide a signal once the cuff is correctly positioned over the endoscope probe. According to some embodiments, at least one of the longitudinal bores of the cuff is configured for passage of imaging means.

According to some embodiments, the cuff has a first collapsed configuration. According to some embodiments, in its collapsed configuration the outer diameter of the cuff encaging/engaging the endoscopic probe is below 15 mm, below 14 mm, below 13 mm, below 12.5 mm, below 12 mm, below 11.5 mm, below 11 mm, below 10.5 mm or below 10 mm. Each possibility is a separate embodiment. According to some embodiments, the outer diameter of the endoscope probe with the cuff in its collapsed configuration is increased by less than 2%, less than 3%, less than 5%, less than 10%, less than 15%, less than 20%, less than 25% or less than 30% as compared to the outer diameter of the endoscopic probe and/or shaft itself. Each possibility is a separate embodiment. According to some embodiments, the circumference of the cuff may be adjusted to fit endoscope probes of various sizes and/or shapes. For example, some cuffs may be provided with a circumference configured to fit endoscopes used in adults, while others are made to fit pediatric use.

According to some embodiments, in the expanded configuration, passage of one or more endoscopic accessory devices through the longitudinal bores is possible.

According to some embodiments, the cuff may be self-expandable. According to some embodiments, the cuff may be configured to expand (automatically) once the probe reaches its target location, for example due to the temperature and/or humidity prevailing at the target location. According to some embodiments, the cuff may be made of or include a shape memory material such as a memory shape alloy or a memory shape polymer. Each possibility is a separate embodiment. According to some embodiments, the memory shape material may be configured to cause expansion of the cuff once reaching the target location.

Additionally or alternatively, the cuff may be expanded “on-the fly” i.e. by insertion/when passing a guide, endoscopic accessory device, medical instrument, imaging probe, and or therapeutics through the longitudinal bores.

Additionally or alternatively, the cuff may be expanded by activating an expansion mechanism. According to some embodiments, the expansion mechanism may be a pump causing the cuff to expand by inflation. According to some embodiments, the expansion mechanism may be a specialized net with an inflatable balloon or other instrument or which is collapsed during insertion, but which may be expanded on demand.

According to some embodiments, each of the one or more longitudinal bores may be separately expanded, for example only when an endoscopic accessory device is introduced through the particular bore. According to some embodiments, all of the bores may be expanded together for example as a result of activation of an expansion mechanism.

According to some embodiments, expansion of the cuff increases the outer diameter of the cuff while leaving the inner diameter of the cuff essentially unaffected, thereby increasing the diameter of the at least one longitudinal bore. As a non-limiting example, passage of an accessory device through the bore may cause the roof of the bore to be lifted/stretched, thereby increasing the diameter thereof. As a non-limiting example, expansion of the bore may be achieved by inserting and inflating an inflatable balloon dedicated to the purpose of expanding the bore.

According to some embodiments, the transition between collapsed and expanded bores may be partial, segmental and optionally reversible. According to some embodiments, the bore may be expanded only along a part of the length thereof. The extended part may change over time and at different segments along the endoscopic shaft. For example, when extracting a large polyp, a short segment of the bore (coincident with the position of the polyp) may be wider than other parts of the same bore. Furthermore, the diameter may change along with the extraction of the polyp.

According to some embodiments, the longitudinal bore is positioned between the inner and outer surfaces of the cuff at a position closer to the outer surface than to the inner surface. As a result, the outer surface surrounding the bore will readily be stretched during expansion, whereas the inner surface is left essentially unaffected.

According to some embodiments, the term “endoscopic accessory device” may refer to any medical instrument suitable for passage through the longitudinal bores including, but not limited to: guide-wires, tubes, stents, clips, snares, biopsy forceps, grabbers, snippers, polyp retrievers, needles, graspers, nets, staplers, band ligation instruments, cutting, coagulation, radiofrequency ablation instruments, imaging probes, irrigation tools or any other tubing, endoscopic device, medical instrumentation or foreign bodies introducible through the longitudinal bore. Each possibility is a separate embodiment. According to some embodiments, the longitudinal bore may be configured to allow passage of an endoscopic device additional to the endoscopic device over which the cuff is draped.

According to some embodiments, the cuff resumes its collapsed configuration when the one or more endoscopic accessory devices are pulled out of the longitudinal bores. According to some embodiments, the cuff may resume its collapsed configuration as a result of the backward movement of the probe, i.e. as a result of its retrieval. According to some embodiments, the cuff may resume its collapsed configuration as a result of the backward movement of the endoscopic accessory device, i.e. as a result of its withdrawal. According to some embodiments, the cuff may resume its collapsed configuration due to deactivation of the expansion mechanism and/or due to activation of a collapse mechanism, e.g. deflation of the cuff or collapsing of the inner structure.

According to some embodiments, the cuff may be made of a semi-elastic or elastic material. According to some embodiments, the semi-elastic/elastic material causes the cuff material to be stretched when an endoscopic accessory device is passed through the bore thereby expanding the diameter of the bore. According to some embodiments, the semi-elastic/elastic material may allow the bore to assume a diameter suitable and/or required for the work therein. For example, according to some embodiments, the bore diameter may be further expanded when a large polyp is retrieved therethrough. According to some embodiments, the expansion may be local, such that an enlarged diameter is achieved along the retrieval path, at the location of the polyp.

According to some embodiments, the cuff is configured to facilitate medical procedures from being performed within the one or more longitudinal bores. According to some embodiments, the cuff is configured to facilitate medical procedures from being performed within the lumen/body cavity of the target organ, within the wall of the target organ/body cavity or within a target area outside the lumen/body cavity (through which the endoscope and cuff is passed through, such as, but not limited to, peritoneal cavity, pleural cavity, mediastinum, oropharynx, and retroperitoneum).

According to some embodiments, the at least one longitudinal bore may include a smoothing layer configured to allow essential friction free and/or smooth passage of endoscopic accessory devices therethrough. According to some embodiments, the additional layer may be an integral part of the cuff, may be provided with the cuff or applied on the cuff prior to use. Each possibility is a separate embodiment.

According to some embodiments, the cuff may include a plurality of pores opening into the at least one longitudinal bore. According to some embodiments, the plurality of pores contain/hold a smoothening fluid. The diameter of the pores may be such that the smoothening fluid is confined within the pores when the cuff is in its collapsed configuration. According to some embodiments, when the cuff assumes its expanded configuration the diameter of the pores is increased due to the stretching of the cuff material. In some embodiments, the pores are open at the baseline. Such pores can be used for application of material during the procedure, including, but not limited to, fluid irrigation, bowel cleansing agents, medications (such as, but not limited to, anti spasmodic, lubricants and dyes).

According to some embodiments, the cuff may have an atraumatic distal end. According to some embodiments, the cuff may include an atraumatic guiding tip/cap configured to allow smooth access of the endoscopic probe, covered by the cuff, into tight tissue locations, while avoiding perforation of/harm to the tissue wall. According to some optional embodiments, the guiding tip/cap may be an integral part of the cuff. According to some optional embodiments, the guiding tip/cap may cover the distal end of the cuff during insertion and be configured for removal once a target location has been reached.

According to some embodiments, the at least one longitudinal bores may be sized and shaped to allow withdrawal of large tissue specimens such as, but not limited to, large polyps, tissue excisions, and foreign bodies therethrough without requiring withdrawal of the endoscopic probe. Today retrieval of large polyps after polypectomy requires withdrawal of the endoscopic probe, detachment of the encaged polyp from the probe and reinsertion of the probe for further inspection/treatment. Retrieval of the polyp, through the additional working channel provided by the cuff, thus allows safe retrieval of the polyp while the endoscopic probe remains in position, thereby enabling a faster procedure, causing less discomfort to the patient and with diminished risk of complications.

According to some embodiments, the cuff may, e.g. a priori, include a polyp retriever device in at least one of the longitudinal bores. According to some embodiments, the polyp retriever device may include an inflatable balloon configured to enlarge the bore prior to retrieving the polyp therethrough. This may ease the entrance and passage of the polyp through the bore and prevent the polyp from being torn in the process. According to some embodiments, the polyp retriever may be an integral feature of the cuff (e.g. slidingly attached to the cuff). According to some embodiments, the polyp retriever may be supplied with the cuff, but as a separate element. According to some embodiments, the polyp retriever may be a stand-alone element, sized and shaped to be used with the cuff.

According to some embodiments, the cuff may include a handle configured to be attached to, draped over or otherwise secured to the proximal end of the endoscopic probe. According to some embodiments, the endoscopic probe may include a feature configured to secure the cuff handle thereto or thereon. According to some embodiments, the cuff handle may include a feature configured to secure the cuff handle to an existing element present on conventional endoscopic probes, such as to the handle of the endoscope probe itself. According to some embodiments, the cuff handle may include at least one access port (also referred to herein as entry channels), the at least one access port configured to provide access of one or more endoscopic accessory devices to the cuff's longitudinal bores.

According to some embodiments, the cuff handle may include a large opening, coextensive with a longitudinal bore sized and shaped for retrieval of large content, such as, but not limited to, polyps, tissue biopsies and foreign bodies. The large opening is thus configured to allow withdrawal of the large content collected through a dedicated longitudinal bore. According to some embodiments, the large opening may also be configured to allow insertion of accessory equipment too large to be inserted through an access port. According to some embodiments, the large opening may also be configured to allow insertion of tubes such as irrigation tubes, allowing adequate and comfortable irrigation without interfering with the medical procedure performed. In some embodiments, irrigation may be performed through the cuff's channels directly, without additional tube insertion. According to some embodiments, the large opening may advantageously be covered by a hood configured to ensure a clean environment, for example by preventing expulsion of fluid, gas, odor and/or bowel content during the procedure. As used herein, the term “hood” and “cover” may be used interchangeably and may refer to a lid or other element configured to reversibly cover the large opening. According to some embodiments, the accessory equipment may be mounted on the hood's framework using a specialized compatible mount, so as to prevent fluid, content and gas leakage when the hood is removed.

According to some embodiments, the cuff may be configured to circumferentially cover the endoscopic probe along its entire length. According to some embodiments, the cuff may be configured to partially cover the endoscopic probe. According to some embodiments, the cuff may be configured to circumferentially cover the endoscopic probe along part of its length while other parts are partially covered.

According to some embodiments, the cuff may be configured to circumferentially cover the endoscopic probe along its entire length while it's proximal part, including the inlets of its working channels are held separate from the endoscope handle, to allow two operators to work simultaneously. According to some embodiments, the cuff may be configured to be anchored to a separate handle to allow simultaneous use by two operators.

According to some embodiments, the cuff may include a fixed portion, configured for attachment to the proximal end of the endoscope probe, and a rotatable portion configured to allow the underlying endoscope's maneuverability.

According to some embodiments, the opening of the additional working channels will be marked and or numbered based on the respective location of the channels' openings.

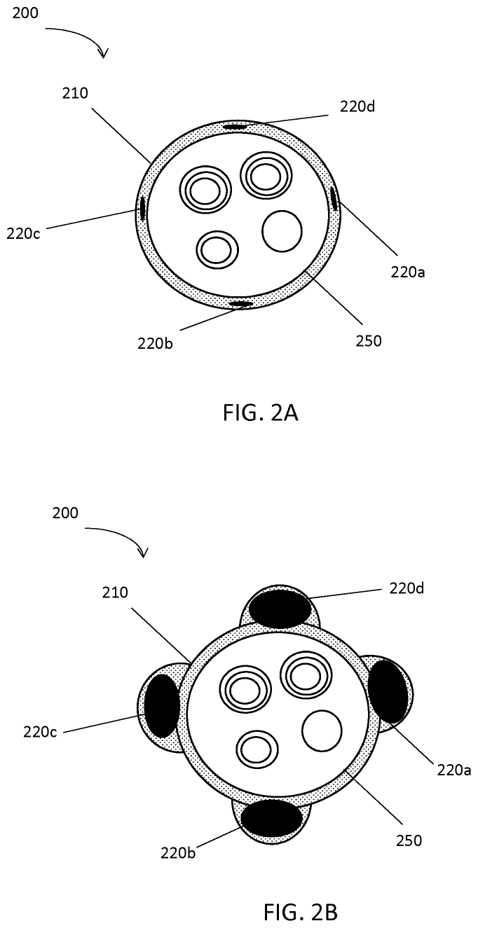

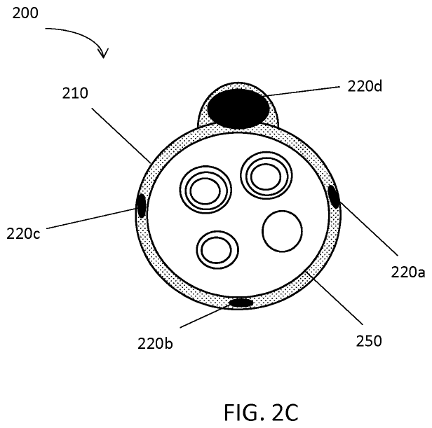

Reference is now made to FIG. 2A, which shows a front view of a distal end of an endoscope assembly 200 including an endoscope cuff 210 draped around an endoscope probe 250 in its collapse configuration; according to some embodiments. Endoscope cuff 210 includes a plurality of longitudinal bores (here shown as four longitudinal bores 220a-220d. In the collapsed configuration, longitudinal bores 220a-220d are collapsed such that the diameter of endoscope probe 250 covered by endoscope cuff 210 is only slightly increased vis-à-vis the diameter of endoscope probe 250, thereby ensuring relatively easy entry of endoscope assembly 200 into a body cavity (not shown) of a patient. Advantageously, endoscope cuff 210 may be made of a single one-piece material, thereby obviating the need for joining sheaths of materials. According to some embodiments, endoscope cuff 210 may include a lubricious material on its outer surface, the lubricious material configured to provide essentially smooth insertion of endoscope assembly 200 into the patient's body cavity. According to some embodiments, endoscopic cuff 210 may be tapered at its distal end, such that the circumference of endoscope cuff 210 is smaller than the circumference of the distal end of endoscope probe 250. Additionally or alternatively, endoscope cuff 210 may include an attachment mechanism configured to grasp the distal end of endoscope probe 250 thereby preventing retraction/folding back of endoscope cuff 210 relative to endoscope probe 250, during insertion.

Reference is now made to FIG. 2B, which shows a front view of a distal end of an endoscope assembly 200 including an endoscope cuff 210 draped around an endoscope probe 250 in its expanded configuration; according to some embodiments. In the expanded configuration, longitudinal bores 220a-220d are expanded to provide working channels allowing passage of endoscopic accessory devices, as essentially described herein. Longitudinal bores 220a-220d are here shown to have essentially identical sizes and shapes. However, alternative configurations in which one (or more) of longitudinal bores 220a-220d has a different size and/or shape can also be envisaged as is within the scope of this disclosure. Advantageously, longitudinal bores 220a-220d may be expanded only when endoscopic assembly 200 reaches its target location so as not to interfere with the introduction of endoscope assembly 200 into the patient's body cavity. According to some embodiments, expansion of longitudinal bores 220a-220d may be obtained as a result of insertion and passage of endoscopic accessory devices (or insertion catheters containing same) therethrough and may collapse with the withdrawal of the accessory devices. Additionally or alternatively, the expansion of longitudinal bores 220a-220d may be by activating an expansion mechanism (e.g. inflating a balloon within the bore), as essentially described herein.

Reference is now made to FIG. 2C, which shows a front view of a distal end of an endoscope assembly 200 including an endoscope cuff 210 draped around an endoscope probe 250 in a partially expanded configuration; according to some embodiments. In this configuration, only longitudinal bore 220d is expanded (e.g. due to passage of an endoscopic accessory device therethrough), whereas longitudinal bores 220a-220c remain collapsed. Advantageously, selective expansion of a subsection of longitudinal bores (e.g. 2 out of four, 1 out of three etc.) enables working with an endoscope probe assembly having the smallest possible diameter, consistent with the number of working channels needed to perform the medical procedure.

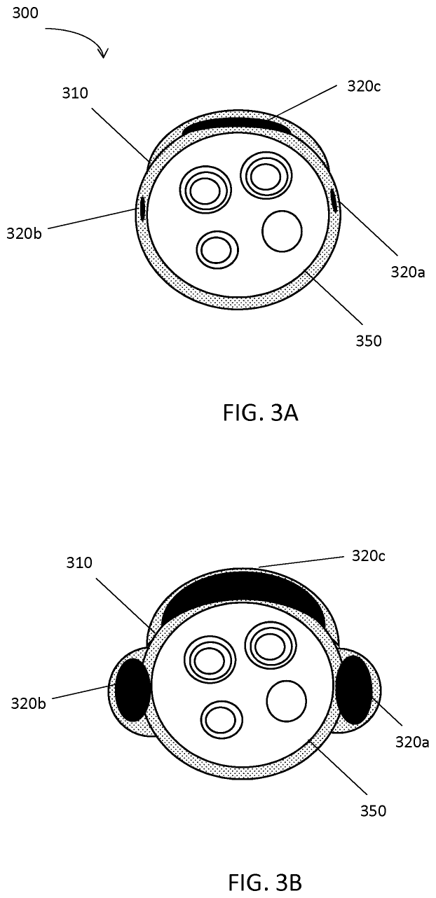

Reference is now made to FIG. 3A, which shows a front view of a distal end of another exemplary endoscope assembly 300 including an endoscope cuff 310 draped around an endoscope probe 350 in its collapse configuration; according to some embodiments. Endoscope cuff 310 includes a plurality of longitudinal bores (here shown as three longitudinal bores 320a-320c. In the collapsed configuration, longitudinal bores 320a-320c are collapsed such that the diameter of endoscope probe 350 covered by endoscope cuff 310 is only slightly increased vis-à-vis the diameter of endoscope probe 350, thereby ensuring relatively easy entry of endoscope assembly 300 into a body cavity (not shown) of a patient. The longitudinal bores 320a-320c of endoscope cuff 310 are here depicted as being of different size and shapes. Longitudinal bore 310c is wider than longitudinal bores 320a and 320b thereby providing a larger working channel when in its expanded configuration, as further elaborated below. Advantageously, endoscope cuff 310 may be made of a single one-piece material, thereby obviating the need for joining sheaths of materials. According to some embodiments, endoscope cuff 310 may include a lubricious material on its outer surface, the lubricious material configured to provide essentially smooth insertion of endoscope assembly 300 into the patient's body cavity. According to some embodiments, endoscopic cuff 310 may be tapered at its distal end, such that the circumference of endoscope cuff 310 is smaller than the circumference of the distal end of endoscope probe 350. Additionally or alternatively, endoscope cuff 310 may include an attachment mechanism (not shown) configured to grasp the distal end of endoscope probe 350 thereby preventing retraction/folding back of endoscope cuff 310 relative to endoscope probe 310, during insertion.

Reference is now made to FIG. 3B, which shows a front view of a distal end of an endoscope assembly 300 including an endoscope cuff 310 draped around an endoscope probe 350 in its expanded configuration; according to some embodiments. In the expanded configuration, longitudinal bores 320a-320c are expanded to provide working channels allowing passage of endoscopic accessory devices, as essentially described herein. Longitudinal bores 320a-320c are different in size and shape, in that longitudinal bore 320c is larger and wider than longitudinal bores 320a and 320b. The enlarged size and shape of longitudinal bore 320c is particularly suitable for enabling extraction of large content such, as but not limited to, polyps, tissue biopsies and foreign bodies therethrough. Advantageously, longitudinal bores 320a-320c may be expanded only when endoscopic assembly 300 reaches its target location so as not to interfere with the introduction of endoscope assembly 300 into the patient's body cavity. According to some embodiments, expansion of longitudinal bores 320a-320c may be obtained as a result of insertion and passage of endoscopic accessory devices (or insertion catheters containing same) therethrough and may collapse with the withdrawal of the accessory devices. Additionally or alternatively, the expansion of longitudinal bores 320a-320c may be obtained by activating an expansion mechanism (e.g. inflating a balloon within the bore), as essentially described herein.

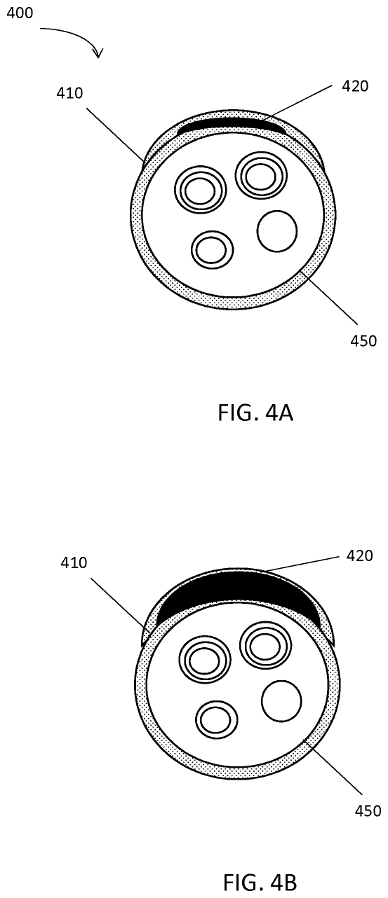

Reference is now made to FIG. 4A, which shows a front view of a distal end of an endoscope assembly 400 including an endoscope cuff 410 draped around an endoscope probe 450 in its collapse configuration; according to some embodiments. Endoscope cuff 410 includes a single longitudinal bore 420. In the collapsed configuration, longitudinal bore 420 is collapsed such that the diameter of endoscope probe 450 covered by endoscope cuff 410 is only slightly increased vis-à-vis the diameter of endoscope probe 450, thereby ensuring relatively easy entry of endoscope assembly 400 into a body cavity (not shown) of a patient. According to some embodiments, endoscope cuff 410 may be made of a single one-piece material, thereby obviating the need for joining sheaths of materials. According to some embodiments, endoscope cuff 410 may include a lubricious material on its outer surface, the lubricious material configured to provide essentially smooth insertion of endoscope assembly 400 into the patient's body cavity. According to some embodiments, endoscopic cuff 410 may be tapered at its distal end, such that the circumference of endoscope cuff 410 is smaller than the circumference of the distal end of endoscope probe 450. Additionally or alternatively, endoscope cuff 410 may include an attachment mechanism (not shown) configured to grasp the distal end of endoscope probe 450 thereby preventing retraction/folding back of endoscope cuff 410 relative to endoscope probe 450, during insertion.

Reference is now made to FIG. 4B, which shows a front view of a distal end of an endoscope assembly 400 including an endoscope cuff 410 draped around an endoscope probe 450 in its expanded configuration; according to some embodiments. In the expanded configuration, longitudinal bore 420 is expanded to provide a working channel sized and shaped to allow passage of endoscopic accessory devices, as well as extraction of large content such as, but not limited to, polyps, tissue biopsies and foreign bodies as essentially described herein. Advantageously, longitudinal bore 420 may be expanded only when endoscopic assembly 400 reaches its target location so as not to interfere with the introduction of endoscope assembly 400 into the patient's body cavity. According to some embodiments, expansion of longitudinal bore 420 may be obtained as a result of insertion and passage of accessory devices (or insertion catheters containing same) therethrough and may collapse with the withdrawal of the accessory devices. Additionally or alternatively, the expansion of longitudinal bores 420 may be achieved by activating an expansion mechanism (e.g. inflating a balloon within the bore), as essentially described herein.

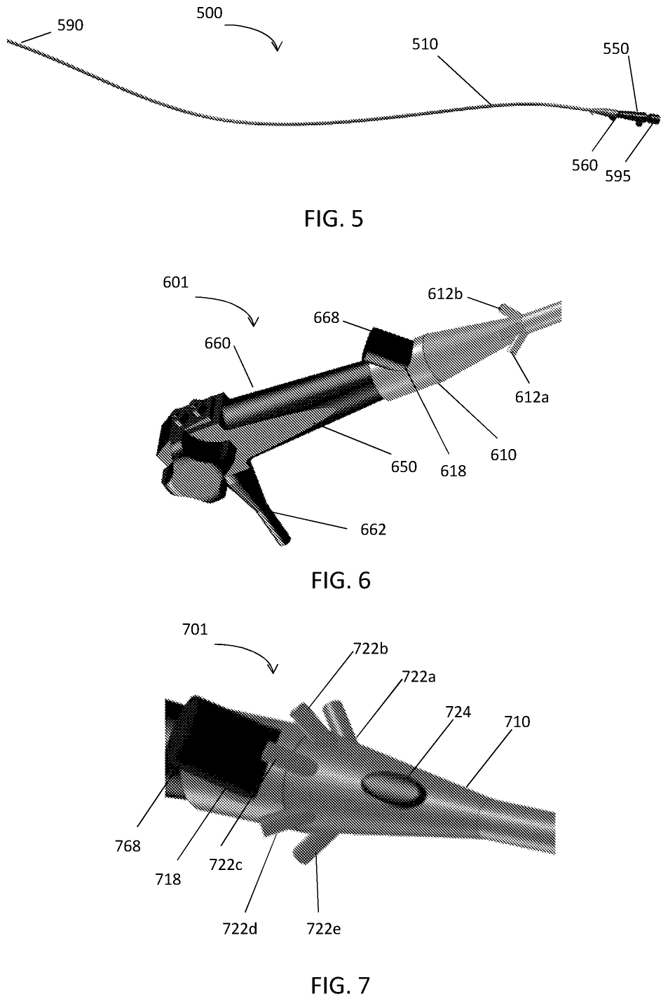

Reference is now made to FIG. 5, which shows a side view of an endoscope assembly 500 including an endoscope cuff 510 draped around an endoscope probe 550, according to some embodiments. As shown, endoscope cuff 510 may cover endoscope probe 550 essentially along its entire length, i.e. from endoscope handle 560 to distal end 590 of endoscope assembly 500. However, other configurations in which endoscope cuff 510 covers endoscope probe 550 along only part of its length may also be envisaged and is within the scope of this disclosure. Proximal end 595 of endoscope assembly 500 may include a connector (not shown) configured to connect endoscope assembly 500 to a main processor. According to some embodiments, cuff 510 may be configured to allow attachment to a different handle while still mounted on the scope's shaft. This may enable a second operator also to perform procedures.

In some embodiments, some of the additional working channels will be functionally attached to the main endoscope handle, and some to a separate handle. The hood of the large opening is anchored to the main endoscope handle in some embodiments, and to a separate handle in others.

In some embodiment, the cuff encompasses the entire circumference of the endoscopic shaft, in others the cuff encompasses a part of the circumference. According to some embodiments, the cuff may be configured to partially cover the endoscopic probe. According to some embodiments, the cuff may be configured to circumferentially cover the endoscopic probe along part of its length while other parts are partially covered.

Reference is now made to FIG. 6, which shows a side view of a proximal end 601 of an endoscope assembly (such as endoscope assembly 500) including endoscope cuff 610 draped around handle 660 of an endoscope probe 650, according to some embodiments. Endoscope cuff 610 includes a proximal attachment element, here an aperture 618, configured to secure endoscope cuff 610 to endoscope probe 650, here bypassing bulky portion 668 of endoscope probe 650 through aperture 618. However, other proximal attachment mechanisms and/or elements can also be envisaged, as essentially described herein, and are within the scope of this disclosure.

Reference is now made to FIG. 7, which shows a side view of a proximal end 701 of an endoscope cuff 710 draped around an endoscope probe (not shown), according to some embodiments. Endoscope cuff 710 includes a proximal attachment element, here an aperture 718, configured to secure endoscope cuff 710 to the endoscope probe, here bypassing bulky portion 768 of the endoscope probe through aperture 718. However, other proximal attachment mechanisms and/or elements can also be envisaged, as essentially described herein, and are within the scope of this disclosure. Proximal end 701 of endoscope cuff 710 includes a plurality of entry channels, here depicted as five entry channels 722a-722e. Entry channels 722a-722e are each configured to allow access of endoscopic accessory devices to a respective longitudinal bore (not shown—e.g. similar to longitudinal bores 320a-320d). Proximal end 701 of endoscope cuff 710 further includes a large opening 724 (preferably covered by a hood) configured to allow withdrawal of large content, such as, but not limited to, polyps, tissue biopsies and foreign bodies collected through a dedicated longitudinal bore (not shown—e.g. a longitudinal bore similar to longitudinal bore 320c. Large opening 724 may also allow mounting and insertion of accessory equipment too large to be inserted through entry channels 722a-722e. Large opening 724 may advantageously be covered so as to ensure a clean environment, for example by preventing irrigation liquids being tossed on the operator. In some embodiments, the cuff overlying the proximal part of the endoscopic shaft (distal to the handle) may be flexible to allow rotation (needed in certain endoscopic models to allow modulation of scope rigidity).

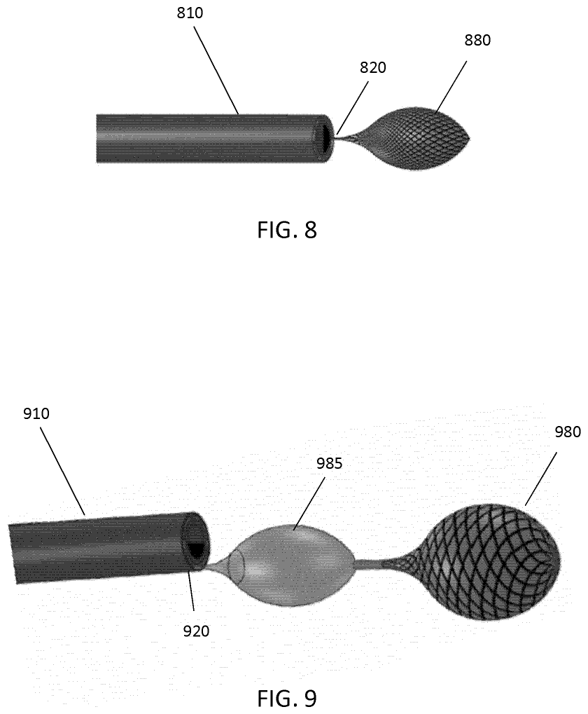

Reference is now made to FIG. 8, which shows the distal end of an endoscope cuff 810 including a polyp retriever 880; according to some embodiments. According to some embodiments, polyp retriever 880 may be an integral part of endoscope cuff 810 i.e. may be inserted in advance to at least one of the longitudinal bore 820 of cuff 810. According to some embodiments, polyp retriever 880 may be supplied with endoscope cuff 810, but as a separate element. According to some embodiments, polyp retriever 880 may be a stand-alone element sized and shaped to be used with endoscopic cuff 810. According to some embodiments, longitudinal bore 820 may be sized and shaped to allow withdrawal therethrough of large polyps after polypectomies, without requiring withdrawal of the endoscope probe over which endoscope cuff 810 is draped. Today retrieval of large polyps after polypectomy requires withdrawal of the endoscope probe, detachment of the encaged polyp from the probe and reinsertion of the probe for further inspection/treatment. Retrieval of the polyp through longitudinal bore 820, on the other hand allows safe retrieval of polyps without requiring withdrawal of the entire endoscope probe, thereby enabling a faster procedure, causing less discomfort to the patient and with diminished risk of complications.

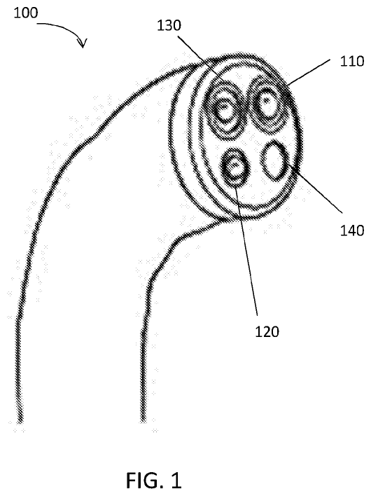

Reference is now made to FIG. 9, which shows the distal end of an endoscope cuff 910 configured to be draped around an endoscope probe (such as but not limited to endoscope probe 100 shown in FIG. 1) including a polyp retriever 980; according to some embodiments. Polyp retriever 980 is inserted in or configured for insertion through longitudinal bore 920 of endoscope cuff 910. According to some embodiments, endoscope cuff 910 includes at least one longitudinal bore 920 sized and shaped to allow retrieval there through of large polyps after polypectomy (such as longitudinal bore 920), without requiring withdrawal of the endoscope probe over which endoscope cuff 910 is draped. In addition, polyp retriever 980 includes an inflatable balloon 985, positioned in proximity to polyp retriever 980 and configured to enlarge longitudinal bore 920 prior to withdrawing the polyp therethrough. This may ease on the entrance and passage of the polyp through the bore, through naturally narrow orifices (e.g.—anus), and prevent the polyp from being torn in the process. In some embodiments, the balloon may be located at various locations relative to the net and its guide.

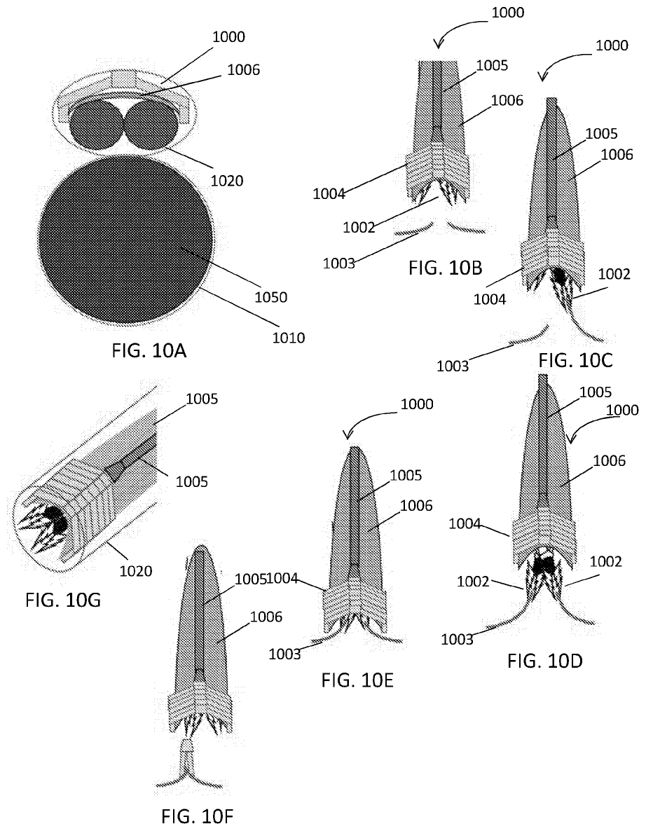

Reference is now made to FIG. 10A through FIG. 10G which show an endoscopic clipping device 1000 configured for use with an endoscope cuff 1010 draped over an endoscope probe 1050; according to some embodiments. FIG. 10A schematically depicts endoscopic clipping device 1000 inserted through a longitudinal bore 1020 of endoscope cuff 1010. Endoscopic clipping device 1000 includes graspers 1002 configured to grasp tissue 1003, such as, but not limited to, opposite sides of a surgical wound, as shown in FIG. 10B and FIG. 10C. Endoscopic clipping device 1000 further includes a clip storage/cartridge/magazine 1004 which preferably includes a plurality of clips (e.g. at least 2, 3, 4, 5, 6 or more clips), which are held open by an underlying cover 1006, which overlies the graspers. Once the opposite sides of tissue 1003 are grasped and approximated to one another, as depicted in FIG. 10D an activating mechanism may be activated, for example due to forward movement of the clip storage/cartridge/magazine by guide 1005, which will push the most distal clip over the underlying cover 1006, as shown in FIG. 10E, thereby releasing a clip from clip storage/cartridge/magazine 1004 and clipping together opposite sides of tissue 1003, as depicted in FIG. 10F. Upon completion of the procedure, endoscopic clipping device 1000 may be withdrawn from longitudinal bore 1020 of endoscope cuff 1010, as depicted in FIG. 10G.

While a number of exemplary aspects and embodiments have been discussed above, those of skill in the art will recognize certain modifications, additions and sub-combinations thereof. It is therefore intended that the following appended claims and claims hereafter introduced be interpreted to include all such modifications, additions and sub-combinations as are within their true spirit and scope.

1. An endoscopic cuff comprising:
an outer surface configured to allow access of the cuff to tight tissue locations;
an inner surface configured to completely or partially circumferentially encase an endoscopic probe;
at least one longitudinal bore formed between the inner and outer surfaces; and
an attachment assembly configured to engage a distal tip of the endoscopic probe, thereby preventing rearward movement of the cuff relative to the endoscopic probe;
wherein the cuff has a first collapsed configuration and a second expanded configuration; wherein in its collapsed configuration the outer diameter of the cuff engaging the endoscopic probe is below 15 mm, wherein the expanded configuration is configured to allow passage of one or more endoscopic accessory devices through the at least one longitudinal bore; and wherein the cuff is formed from a one-piece material thereby obviating a need for joining materials.

2. endoscope of claim 1, wherein the endoscopic cuff is configured partially or completely to encircle the endoscopic probe.

3. The endoscopic cuff of claim 1, wherein the cuff assumes its expanded configuration when one or more endoscopic accessory devices are passed through the at least one longitudinal bore.

4. The endoscopic cuff of claim 3, wherein the cuff resumes its collapsed configuration when the one or more endoscopic accessory devices are pulled out of the at least one longitudinal bore.

5. The endoscopic probe of claim 1, wherein the diameter of the cuff is configured to be enlarged segmentally during passage of the one or more endoscopic accessory devices.

6. The endoscopic cuff of claim 1, wherein the cuff is made from a shape memory alloy or a shape memory polymer.

7. The endoscopic cuff of claim 1, wherein the attachment assembly comprises a tapered portion at a distal tip of the cuff; wherein a circumference of the distal tip is smaller than a circumference of a distal end of the endoscopic probe.

8. The endoscopic cuff of claim 1, wherein the attachment assembly comprises an attachment element configured to grasp or attach to a distal end of the endoscopic probe.

9. The endoscopic cuff of claim 1, wherein in its collapsed configuration the outer diameter of the cuff engaging the endoscopic probe is below 13 mm.

10.-11. (canceled)

12. The endoscopic cuff of claim 1, wherein the at least one longitudinal bore comprises a smoothing layer configured to allow essential smooth passage of endoscopic accessory devices therethrough.

13. The endoscopic cuff of claim 1, wherein the at least one longitudinal bore is angled relative to the endoscope probe, at least along part of its length.

14. The endoscopic cuff of claim 1, wherein the cuff comprises a plurality of pores opening into the at least one longitudinal bore, the plurality of pores containing a smoothening fluid; wherein the diameter of the pores is configured to increase when the cuff assumes its expanded position, thereby causing said smoothening fluid to spread on the wall of the at least one longitudinal bore.

15. (canceled)

16. The endoscopic cuff of claim 1, wherein the at least one longitudinal bore is sized and shaped to allow withdrawal of large tissue specimens, bowel content and/or foreign bodies therethrough, without requiring withdrawal of the endoscopic probe.

17. The endoscopic cuff of claim 1, wherein the at least one longitudinal bore comprises a polyp retriever device.

18. The endoscopic cuff of claim 17, wherein the polyp retriever device comprises an inflatable balloon configured to enlarge at least one longitudinal bore prior to retrieving a polyp, tissue, bowel content or a foreign body therethrough.

19. The endoscopic cuff of claim 17, further comprising a large opening at its proximal end, the large opening configured to allow withdrawal of a polyp, tissue, bowel content and/or a foreign body collected through the at least one longitudinal bore.

20.-22. (canceled)

23. The endoscopic cuff of claim 1, further comprising a handle anchor having at least one access port, the at least one access port configured to provide access of the one or more endoscopic accessory devices to the at least one longitudinal bore.

24. The endoscopic cuff of claim 1, wherein expansion of the cuff increases an outer diameter of the cuff while leaving an inner diameter of the cuff essentially unaffected, thereby increasing the diameter of the at least one longitudinal bore.

25. (canceled)

26. The endoscopic cuff of claim 1, comprising at least two longitudinal bores; wherein at least one of the at least two longitudinal bores is larger than the remainder of the at least two longitudinal bores.

27.-29. (canceled)

30. An endoscopic cuff comprising:
an outer surface configured to allow access of the cuff to tight tissue locations;
an inner surface configured to circumferentially encase an endoscopic probe; at least one longitudinal bore formed between the inner and outer surfaces;
an attachment assembly configured to engage the distal tip of the endoscopic probe, thereby preventing rearward movement of the cuff relative to the endoscopic probe; and
a polyp retriever device configured for insertion through the at least one longitudinal bore, the polyp retriever device comprising an inflatable balloon configured to enlarge the at least one longitudinal bore and/or narrow orifices prior to retrieving a polyp, tissue, and/or a foreign body therethrough;
wherein the cuff has a first collapsed configuration and a second expanded configuration.

31.-41. (canceled)
